(19) United States
(12) Patent Application Publication
(10) Pub. No.: US 2008/0306245 A1
(43) Pub. Date: Dec. 11, 2008

(54) Method For The Production Of Albumen Conjugates With Non-Steroidal Antirheumatic Drugs (Nsar)

(75) Inventors: Hannsjorg Sinn, Wiesloch (DE); Marcel Mulbaier, Heidelberg (DE)

Correspondence Address: MORRIS MANNING MARTIN LLP, 3343 PEACHTREE ROAD, NE, 1600 ATLANTA FINANCIAL CENTER, ATLANTA, GA, 30326, US

(73) Assignee: ALBUPHARM HEIDELBERG GMBH & CO. KG, Heidelberg (DE)

(21) Appl. No.: 11/791,584

(22) PCT Filed: Nov. 25, 2005
(86) PCT No.: PCT/EP2005/012638

§ 371 Date: Feb. 7, 2008

(30) Foreign Application Priority Data
Nov. 26, 2004 (DE) 10 2004 057 196.1

(51) Int. Cl. C07K 14/76

(52) U.S. Cl. 530/362

(57) ABSTRACT

The present invention concerns NSAR (non-steroidal antirheumatic agents)-protein conjugates and in particular NSAR-albumin conjugates, processes for their production and their use as pharmaceutical preparations especially for treating inflammations.

The present invention concerns NSAR (non-steroidal antirheumatic agents)-protein conjugates and in particular NSAR-albumin conjugates, processes for their production and their use as pharmaceutical preparations especially for treating inflammation.

Non-steroidal antirheumatic agents (NSAR), also referred to NSAIDs (non-steroidal anti-inflammatory drugs) have previously been used in low-molecular weight forms to treat inflammatory processes. A disadvantage of the previously used treatments using NSAR is in particular the short residence time of the substances used in the circulation so that they only have a very narrow time window to deploy their activity and, moreover, only a small proportion of the administered medicament reaches the target site. This results in undesired side-effects which lead to problems especially when administered for a long time. Thus a substantial proportion of the active substances is taken up by healthy organs where they cause partial or severe damage. Observed side-effects are for example complaints in the region of the gastro-intestinal tract and an inhibition of blood coagulation. Furthermore, concentration disorders and headache were also observed. Finally in some cases there were even allergic reactions, a promotion of asthmatic attacks (in particular in the case of acetylsalicylic acid) and a reduction of white and/or red blood cells.

EP 0 630 263 describes conjugates consisting of an active substance containing carboxyl groups, a spacer and a carrier. The active substance which can be a non-steroidal antirheumatic agent (NSAR) such as for example naproxen, is coupled via an ester bond to the spacer which in turn is linked by a covalent bond to the carrier. (Poly)-α-hydroxy acids come into consideration as spacers which serve to release the active substance to the target tissue in a controlled manner and proteins such as isozyme, cytochrome C and aprotein come into consideration as carriers. However, a disadvantage of these conjugates is that although the active substance is enzymatically cleaved from the spacer at the site of action in the body, the carrier protein initially continues to remain in the body without serving as an energy supplier for the cells associated with inflammatory processes which have a high turnover of plasma proteins. Furthermore, the use of a spacer in addition to the active substance and carrier involves a considerable amount of work and thus increases the costs without being of additional pharmacological utility.

WO 98/00172 discloses pharmaceutical compositions which are intended to specifically release active substances at the target site in the body. They comprise microbubbles which are encased by a protein coat and which are the actual transporters for the active substances. The protein coat preferably consists of human serum albumin and they can for example contain naproxen as the active substance. The microbubbles are formed by means of ultrasonication. However, WO 98/00172 does not disclose that there is a direct covalent bond between the albumin and the active substance. Although it refers to the high specificity of the composition, a longer retention time of the substance in the body which would be based on a direct covalent bond is not disclosed.

Kostiainen et al. (Journal of Chromatography (1993), 647(2), 361-365) disclose the investigation of naproxen-lysozyme conjugates by means of capillary electrophoresis and mass spectroscopy wherein the naproxen is coupled covalently by an amide bond to the lysine group in lysozyme. However, no information whatsoever is given on the pharmacological effect of the conjugate, the extent to which the lysozyme is loaded with the active substance naproxen, the production process for the conjugate etc. Thus the document Kostiainen et al. only concerns physical investigations on the disclosed conjugate.

Manoharan et al. (Chem. Bio. Chem. (2003), 3(12), 1257-1260) discuss the binding affinity of antisense oligonucleotides to serum albumin and find that the binding of ibuprofen to the oligonucleotides has an amplifying effect on the affinity of the oligonucleotides to human serum albumin. Thus, the document does not concern a covalent binding of the active substance ibuprofen to human serum albumin and it also does not mention pharmacological and native properties of a NSAR-HSA conjugate.

Lister et al. (Am. J. Med. 1993 Aug. 9; 95(2A):2S-9S) only disclose a clinical comparison of anti-inflammatory non-steroidal antirheumatic agents such as diclofenac, naproxen or ibuprofen and their effect in the treatment of osteoarthritis or rheumatoid arthritis. A conjugation of these active substances to albumin and a concomitant increase in bioavailability of the NSAR in the body are not disclosed.

Zia-Amirhosseini et al. (Biochem. J. (1995), 311, 431-435) describe the synthesis and mass spectrometric characterization of human serum albumin which is modified by covalent binding to tolmetin. Pharmacological properties of the conjugate are, however, not stated.

Melgert et al. (Cells of the Hepatic Sinusoid (1997), Vol. 1996, 6, 389-390) describe a naproxen-HSA conjugate in which naproxen and HSA are covalently linked together. It was the aim of the inventors to deliver naproxen into Kupfer's cells or epithelial cells of the liver more efficiently than is possible with the free low-molecular weight active substances. The conjugates are used to treat acute /chronic liver inflammation. A major disadvantage of the experiments disclosed in Melgert et al. and Zia-Amirhosseini et al. is the disclosed high quantity ratio of NSAR to albumin because at such a high loading it can by no means be assumed that the protein is active. Thus a denatured product that can no longer be administered is present. Thus it can no longer be ensured that the active substance is released directly at the site of action and that it is thus substantially free of side-effects.

One object of the present invention was therefore to provide NSARs in a form in which the difficulties occurring in the state of the art can be overcome and which enables a specific uptake into inflamed tissue while at the same time having a long half-life in the organism.

This object is achieved according to the invention by an NSAR-protein conjugate which comprises an NSAR and a protein. The NSAR-protein conjugate is particularly preferably an NSAR-albumin conjugate comprising an NSAR and albumin which is characterized in that the NSAR is directly covalently bound to the albumin and that the molar ratio of NSAR:protein is 2:1 to 0.1:1.

Direct coupling of NSARs to proteins and in particular to carrier proteins hides the low-molecular weight active substances which are rapidly removed from the body, from the elimination and capture mechanisms of the body and a long half-life in the body is achieved. This allows small amounts of active substance to be administered and thus almost completely eliminates side-effects that may occur.

Toxic effects on healthy tissue or organs are practically not observed because normal healthy cells have no reason to take up proteins. In contrast proteins and in particular albumin are taken up by cells associated with inflammatory processes and thus lead to an accumulation of active substance in these cells.

A direct covalent coupling of the NSAR to the carrier means that the NSAR is bound to the transport protein by a linker-free or spacer-free bond. The NSAR is preferably covalently bound to the protein by an acid amide bond which is formed from a carboxyl group of the NSAR and an amino group, preferably a lysine group of the protein. The protein that is preferably used in the conjugates according to the invention is albumin, in particular serum albumin and most preferably human albumin or human serum albumin (HSA). Basically a protein is preferably used which is native to the patient for which the conjugate is intended. A native protein is understood as a protein which is derived from the same species as the species to which the protein is administered. This for example means that in the case of administration to humans, human proteins are used and in the case of administration to mice corresponding murine proteins etc. are used.

Human albumin is an endogenous, ubiquitously distributed and non-immunogenic protein. It has a molecular weight of about 68 kDa and is thus not excreted by the kidney. Albumin constitutes approximately 60% of the total amount of plasma protein. In the healthy organism it has among others transport functions for many substances and in an acute emergency serves as a reserve energy carrier which is available everywhere and at any time in the body. Under normal conditions it is not taken up by healthy tissue. In contrast cells associated with inflammatory processes have a high turnover of proteins, in particular of plasma proteins and mainly of albumin. T his leads to a degradation of albumin in the inflamed target cells for which the protein serves as an energy supply and the active substance is released. This means that due to the coupling according to the invention of NSAR to proteins, in particular to albumin, a specific uptake and thus an accumulation of the active substance at the target site can be achieved. This accumulation allows on the one hand, overall lower doses to be used and, on the other hand, hardly any or no side-effects are observed because the conjugate is not taken up by the remaining healthy body tissue.

The biokinetic behaviour of the conjugates according to the invention is solely determined by the macromolecule albumin but not by the low-molecular weight NSAR. The NSAR is preferably coupled to the carrier protein, for example albumin without impairing its biologically active character. The active substance is particularly preferably covalently coupled to the carrier protein. Furthermore, the covalent coupling is preferably selected such that it can be cleaved again in pathologically changed tissues so that the biological efficacy of the original medicament is retained and can be utilized.

Thus according to the invention NSAR-protein conjugates and in particular NSAR-albumin conjugates are formed without changing the biological efficacy of the active substance and without loss of the biologically active character of the protein used as a carrier and in particular of the albumin.

A biologically active protein is understood in particular as a non-denatured protein the biological function of which is preserved. The protein preferably has a biological activity of >50%, particularly preferably of >70%, in particular preferably of >80% and most preferably of >90% based on the activity of natural albumin. In the conjugates the molar ratio of active substance to carrier protein is 2:1 to 0.1:1, preferably 1.5:1 to 0.2:1 and in particular 1.1:1 to 0.5:1. Thus for example albumin still exhibits biologically active behaviour when loaded 1:1 with an NSAR.

Furthermore, it was found that conjugates according to the invention can be obtained which have no or only a very small proportion of cross-linking during loading. This allows a rapid elimination from the circulation and undesired side-effects can be further avoided. The proportion of dimeric albumin in the conjugates according to the invention is advantageously ≦5% by weight, more preferably ≦3% by weight.

According to the invention NSARs are used as the active substance which are in particular selected from acetylsalicylic acid, diclofenac, indomethacin, naproxen, flufenamic acid, mefenamic acid, tolmetin, ibuprofen, fenoprofen, ketoprofen, acemetacin or niflumic acid.

The conjugates according to the invention and the preferred embodiments described herein can offer in particular the following advantages. The active substances are only released in the region of the inflammatory process, in particular by enzymatic cleavage of the protein. The previously common side-effects which occur when using low-molecular weight NSARs no longer occur because healthy cells do not take up albumin or its conjugates in vivo. The biological half-life of the conjugates is solely determined by the macromolecular protein and in particular albumin. Thus, the initially available concentration of active substance only falls to about 50% of the initial value after approximately 20 days. The protein used according to the invention to form the conjugates preferably has a molecular weight of ≧18,000 Da, more preferably ≧30,000 Da and even more preferably ≧50,000 Da.

The long biological half-life substantially widens the previously very narrow time window of active substance availability without further side-effects occurring.

In a preferred embodiment the conjugate according to the invention is an active ingredient in a pharmaceutical preparation. Such a pharmaceutical preparation in particular has low side-effects and can for example also be administered to ambulant patients. It is preferably administered intravenously.

One dosage unit preferably contains 0.1 to 10 mg active substance NSAR per kilogram body weight per day and in particular 0.5 to 5 mg active substance per kilogram body weight per day. The dose can in particular be chosen to be lower than for conventional treatment with NSARs.

Due to the long half-life of the conjugates in the body it is possible to plan longer time intervals between administrations so that one dosage unit is for example administered every seven days at most.

The conjugates according to the invention are particularly suitable for treating inflammatory processes. In this connection the conjugates according to the invention can be used to treat all inflammatory processes which are also treated with the NSARs alone. Inflammatory processes which can be treated according to the invention are for example rheumatoid arthritis.

Furthermore, it is possible to use the conjugates according to the invention in a combination therapy for example together with other anti-inflammatory agents. Doses of the respective components are further reduced in such a combination therapy.

Another subject matter of the present invention is a process for producing an NSAR-protein conjugate. Particularly preferred within the scope of the present invention is a process for producing an NSAR-albumin conjugate comprising reacting an NSAR with albumin by a direct covalent coupling wherein the molar ratio of NSAR: albumin is 2:1 to 0.1:1.

In this process the low-molecular weight active substance NSAR and the high-molecular weight carrier protein are reacted with one another. The active substance is preferably directly covalently coupled to the carrier molecule for example albumin, without restricting its biologically active character. Coupling has proven to be particularly advantageous in which firstly a succinimidyl ester is formed from the low-molecular weight NSAR and this is subsequently reacted with the protein. A succinimidyl ester of NSAR can for example be produced by reacting low-molecular weight NSAR with a carbodiimide.

For the production of the conjugates that are used according to the invention it is important that the active substance is efficiently covalently coupled to the carrier molecule (i.e. the protein). In particular undesired changes of the carrier protein or/and of the active substance should not occur during the coupling. The conventional activation of organic compounds containing carboxyl groups with dicyclohexyl carbodiimide (DCC) requires more than 12 hours at room temperature or at +4° C. (DP 51 22 210 A1; EP 0 879 604 A1; EP 0 820 308). Moreover, in this process insoluble substances are formed during the activation which already partially precipitate during the activation and partially precipitate when the activated active substance is introduced into an aqueous protein solution and, in order that the conjugate can be administered medically, have to be separated by time-consuming and expensive filtration steps in addition to the actual product purification which are never 100% effective (due to the lipophilic domains in the albumin).

There was therefore a need to provide a process for producing active substance-protein conjugates in which these problems do not occur and in which in particular no water-insoluble by-products are formed.

This object is achieved according to the invention by a process for producing a conjugate in which NSAR and albumin are reacted in the presence of N-(3-dimethylaminopropyl)-N′-ethyl carbodiimide (EDC) as the carbodiimide and N-hydroxysuccinimide.

Surprisingly it was found that the use of EDC especially in the form of a hydrochloride enables an activation of the organic compound containing carboxyl groups and reaction with a carrier protein without formation of water-insoluble by-products which would have to be separated in a time-consuming and costly manner. Intermediate purification steps become redundant in this process and the preparation time and thus also the production costs are substantially reduced. Furthermore, problems that are caused by insoluble substances or by-products when injecting the conjugate into a human or animal body are avoided.

The activation preferably takes place at a temperature of 10 to 100° C., more preferably of 20 to 90° C. and even more preferably of 50 to 75° C. for a reaction time of I minute to 10 hours, more preferably of 20 to 50 minutes. The activated active substance is preferably reacted with the carrier protein at a temperature between 10 and 50° C., in particular between 20 and 40° C.

The carboxyl-containing compound, in particular methotraxate is preferably activated with EDC and N-hydroxysuccinimide in an organic solvent, preferably in dimethyl sulfoxide (DMSO). Other suitable organic solvents are for example dimethylacetamide or dioxan. The activation is preferably carried out with the exclusion of water, in particular in the presence of ≦5% by weight water, more preferably ≦1% by weight water and most preferably under completely water-free conditions. As a result of the activation of the compound containing carboxyl groups with the substances 1-DC and N-hydroxysuccinimide in an organic anhydrous solvent, they do not react with protein e.g. albumin and also do not change its structure.

A major advantage of the production process according to the invention is that the activation reagents that are used i.e. EDC and N-hydroxysuccinimide are very soluble in water. As a result coupling reagents that are not consumed during the reaction can be removed in a simple manner from the product obtained, for example by washing with water. In contrast in the case of the coupling reagents used in the prior for example when dicyclohexyl carbodiimide (DCC) is used a non-separable residue of coupling reagent remains in the conjugate. Thus, when using DCC a non-separable residue of about 13 to 15% by weight of DCC is observed in the conjugate in the case of an NSAR-albumin conjugate which is probably bound to a lipophilic domain in the albumin. This residue can only be detected with the aid of HPLC and can only be preparatively separated in a very laborious manner.

Another preferred aspect of the invention concerns an optimized production process for a conjugate according to the invention comprising reacting an NSAR with albumin by a direct covalent coupling characterized in that an NSAR and albumin are reacted in the presence of N-(3-dimethylaminopropyl)-N′-ethyl carbodiimide as the sole activation reagent.

In a preferred embodiment the NSAR is preferably a cytostatic agent or immuno-suppressant, particularly preferably indomethacin, acetylsalicylic acid, diclofenac, ibuprofen and/or naproxen, and especially preferably indomethacin. The protein is preferably albumin.

It was surprisingly found that the optimized process which does not use N-hydroxysuccinimide or other additional activation reagents has a positive effect on the purification procedure and simplifies the production process. As a result of using EDC for activation without the addition of N-hydrosuccinimide (HSI), the activation time of indomethacin is only substantially less than the 30 minutes required when using HSI. Another advantage of the optimized process is that after adding the activated active substance to the initially added protein, in particular albumin without N-hydroxysuccinimide, it is possible to directly monitor the coupling efficiency. When N-hydroxysuccinimide is used it also has a high UV absorption in the HPLC when the UV measuring cell is adjusted to 280 nm and, due to its retention time of about 11.5 minutes at which other low-molecular weight compounds also appear, interferes with or makes it difficult to directly determine the coupling yield. This means that in many cases the yield can only be determined at the end of the purification of the conjugate. This factor can now be excluded by the optimized process without using N-hydroxysuccinimide. This is also of major advantage for product safety. Another advantage of the optimized process is that the coupling yield is surprisingly on average 98 to 99%.

Thus, the total costs of the respective conjugate are considerably reduced by this simplification of the production.

The conjugates produced by the process according to the invention can be provided for numerous applications and in particular for intravenous administration due to their high purity. Thus, for example when using an NSAR having anti-inflammatory activity, such conjugates can be used advantageously to produce pharmaceutical preparations for treating inflammatory processes and in particular to produce a pharmaceutical preparation to treat rheumatoid arthritis.

The invention is further elucidated by the following example and the attached figures.

FIG. 1 shows a HPLC chromatogram of indomethacin alone; and

FIG. 2 shows the chromatogram of the indomethacin-HSA conjugate produced according to example 1.

### EXAMPLE 1

### Indomethacin-HSA Conjugate

### Starting Substances:

Indomethacin (IMC, SIGMA-ALDRICH-1, Taufkirchen), N-(3-dimethylamino-propyl)-N′-ethylcarbodiimide hydrochloride (EDC, SIGMA-ALDRICH, Taufkirchen), N-hydroxysuccinimide (Sigma-Aldrich, Taufkirchen) and albumin (Göricke, Dessau). Other NSARs such as: acetylsalicylic acid, diclofenac, ibuprofen, naproxen etc. can also be used for conjugation with albumin instead of indomethacin.

### Standard Preparation on a Laboratory Scale:

About 10.5 mg indomethacin (IMC, MW 357.8) is placed together with about 11.3 mg N-(3-dimethylaminopropyl)-N′-ethylcarbodiimide hydrochloride (EDC MW 191.71, molar ratio 1:2) and about 34 mg N-hydroxysuccinimide (NHS, MW 115.9, molar ratio 1:10) in a test tube with an NS 14.5 ground joint and stopper. After adding 1 ml dimethyl sulfoxide (DMSO) the reaction mixture is introduced into a water bath preheated to 65° C. After a reaction time of about 30 min, a clear, colourless solution of indomethacin succinimidyl ester is present which, after cooling to room temperature, is introduced very slowly into a 5% albumin solution. A turbidity is briefly formed at the point where it is poured in which, however, rapidly dissolves again.

The control chromatogram can be prepared directly after the coupling. The coupling yield is above 80%.

The undesired accompanying substances in the end product DMSO, NHS, N-(3-dimethylaminopropyl)-N′-ethylurea and non-bound indomethacin are separated by ultrafiltration (YM 30, Millipore).

### Quality Control (HPLC/SEC):

| | |
|---|---|
| Precolumn: | Reprosil 200 SEC 5 × 4 mm, 5 μm (Dr. Maisch GmbH) |
| Column: | Reprosil 200 SEC 300 × 4.6 mm, 5 μm (Dr. Maisch |
| | GmbH) |
| Eluant: | 0.18 M Na₂HPO₄; pH 7.4; 5% methanol |
| Flow rate: | 0.3 ml/min |
| Pressure: | about 50 b |
| UV-vis: | 280 nm |

### Retention Times:

| | |
|---|---|
| oligomeric albumin fraction | 5.93 min |
| dimeric albumin fraction | 8.19 min |
| monomeric albumin fraction | 8.98 min |
| free indomethacin | 29.45 min |

The proportion of dimeric albumin is about <3%.

1. Use of an NSAR-albumin conjugate comprising a non-steroidal antirheumatic agent (NSAR) and albumin for producing a pharmaceutical agent to treat rheumatoid arthritis,
characterized in that
in the conjugate the NSAR is directly covalently bound to the albumin and the molar ratio of NSAR:albumin is in the range of about 2:1 to 0.1:1, wherein the conjugate is obtainable by reacting an NSAR and albumin in the presence of N-(3-dimethylamino-propyl)-N′-ethylcarbonyldiimide as the sole activation reagent in an organic solvent.

2. The use according to claim 1,
characterized in that
the albumin is human albumin.

3. The use according to claim 1,
characterized in that
the albumin is present in native form.

4. The use according to claim 1,
characterized in that
the NSAR is selected from the group consisting of acetylsalicylic acid, diclofenac, indomethacin, naproxen, flufenamic acid, mefenamic acid, tolmetin, ibuprofen, fenoprofen, ketoprofen, acemetacin and niflumic acid.

5. The use according to claim 1,
characterized in that
the molar ratio of NSAR:albumin is in the range of about 1.1:1 to 0.5:1.

6. A process for the production of a conjugate comprising the reaction of an NSAR with albumin by direct covalent coupling,
characterized in that
an NSAR and albumin are reacted in the presence of N-(3-dimethylamino-propyl)-N′-ethylcarbonyldiimide as the sole activation reagent in an organic solvent,
wherein the molar ratio of NSAR:human albumin is in the range of about 2:1 to 0.1:1.

7. The process according to claim 6,
characterized in that
the NSAR is reacted in an anhydrous organic solvent with N-(3-dimethyl-aminopropyl)-N′-ethylcarbodiimide as the sole activation reagent, is activated by heating and subsequently the NSAR is reacted with human albumin.

8. An NSAR-albumin conjugate comprising an NSAR and albumin that is obtainable by the process according to claim 6.

9. The NSAR-albumin conjugate according to claim 8,
characterized in that
the albumin is human albumin.

10. The NSAR-albumin conjugate according to claim 8,
characterized in that
the albumin is present in a native form.

11. The NSAR-albumin conjugate according to claim 8,
characterized in that
the NSAR is selected from the group consisting of acetylsalicylic acid, diclofenac, indomethacin, naproxen, flufenamic acid, mefenamic acid, tolmetin, ibuprofen, fenoprofen, ketoprofen, acemetacin and niflumic acid.

12. The NSAR-albumin conjugate according to claim 8,
characterized in that
the molar ratio of NSAR:albumin is in the range of about 1.1:1 to 0.5:1.

13. A pharmaceutical agent containing an NSAR-albumin conjugate according to claim 8 as the active substance.
